(19) United States
(12) Patent Application Publication
(10) Pub. No.: US 2004/0034432 A1
(43) Pub. Date: Feb. 19, 2004

(54) Mobile bearing tibial base prosthetic devices employing oxidized zirconium surfaces

(76) Inventors: Dean Hughes, Memphis, TN (US); Christopher P. Carson, Memphis, TN (US); Harsh Gupta, Memphis, TN (US); Gordon Hunter, Memphis, TN (US)

Correspondence Address: Joel R. Petrow, Esq., Smith & Nephew, Inc., 1450 Brooks Road, Memphis, TN, 38116, US

(21) Appl. No.: 10/637,975

(22) Filed: Aug. 8, 2003

Related U.S. Application Data
(63) Continuation-in-part of application No. 10/073,705, filed on Feb. 11, 2002.

(51) Int. Cl. A61F 2/38

(52) U.S. Cl. 623/20.28

(57) ABSTRACT

An orthopedic implant with a diffusion-hardened surface on non-load bearing areas of the implant for interaction with non-load bearing surfaces of a polymeric bio-compatible material, such as UHMWPE (ultra-high molecular weight polyethylene). The orthopedic implant is a mobile-bearing knee prosthetic and system where a coating of oxidized zirconium is formed on the post of the tibial tray of the prosthetic for interaction with an opening of a polymeric tibial insert. The diffusion-hardened surface of the orthopedic implant provides a strengthened post and reduction in wear in the opening of the polymeric insert.

This application is a continuation-in-part of U.S. patent application Ser. No. 10/073,705, filed Feb. 11, 2002.

### BACKGROUND OF THE INVENTION

1. Field of the Invention

The field of this invention relates generally to orthopedic prosthetic devices, and more particularly to mobile bearing knee prosthetic devices employing diffusion-hardened surfaces. The invention relates to a knee implant with a diffusion-hardened surface on non-load bearing, non-joint surfaces of the implant for interaction with a polymeric, bio-compatible material, such as UHMWPE (ultra-high molecular weight polyethylene).

2. General Background of the Invention

U.S. Pat. No. 5,037,438 (the '438 patent) and U.S. Pat. No. 5,180,394 (the '394 patent) to Davidson (which are incorporated herein by reference) recognized that a thin coating of zirconium oxide, nitride, carbide or carbonitride is especially useful on the portions of prosthetics, especially metallic orthopedic implants for load bearing surfaces which are subject to high rates of wear. An example cited is a femoral head of a hip-system prosthesis which engages a counter-bearing surface in an acetabular cup which is often made of a softer material such as ultra-high molecular weight polyethylene. The Davidson '438 and '394 patents further recognized that zirconium oxide and nitride coatings on non-load bearing surfaces of an orthopedic implant that contact tissue provides a barrier between the metallic prosthesis and body tissue which prevents the release of metal ions and corrosion of the implant.

The zirconium oxide or nitride coating provides the prosthesis with a thin, dense, low friction, wear resistant, bio-compatible surface ideally suited for use on articulating surfaces of joint prostheses wherein a surface or surfaces of the joint articulates, translates or rotates against mating joint surfaces. The zirconium oxide or nitride may be employed on the articulating surfaces of femoral and tibial (miniscal bearing) surfaces of knee joints.

Another Davidson patent, U.S. Pat. No. 5,415,704 (the. '704 patent), (which is incorporated herein by reference) further discusses the creation of a diffusion-hardened surface of bio-compatible metallic metals and alloys, suitable for use as material for a medical implant, including in particular, niobium, titanium, and zirconium based alloys. The '704 patent discusses various methods of oxidizing or nitriding metals and alloys to provide a fine oxide or nitride dispersion.

The Davidson patents, however, did not address the issue of a knee prosthetic having a diffusion-hardened surface, such as a zirconium oxide surface, for non-loading bearing surfaces of the prosthetic that contacts non-load bearing surfaces of a second prosthetic. The Davidson patents only addressed load-bearing articulating joint surfaces having a zirconium oxide surface where the load bearing joint surface either articulated against body tissue or against another load bearing joint surface.

U.S. Pat. No. 6,123,728 to Broshnahan et al. and U.S. Pat. No. 6,428,477 to Evans et al., titled Mobile Bearing Knee Prosthesis, (which are both incorporated by reference) discloses an orthopaedic knee component for implanting within a proximal tibia includes a tibial tray having a distally extending stem, a proximal tibial plateau and an annular shaped recess extending into the tibial plateau. The recess has a substantially constant radius of curvature about an axis of rotation. A bearing carried by the tibial tray has an articular bearing surface for engagement with a femoral component. The bearing has an annular shaped projection extending into the recess. The projection and the recess allow pivotal movement of the bearing relative to the tibial plateau about the axis of rotation.

U.S. Pat. No. 6,296,666 to Gardner, titled Mobile Bearing Knee with Center Post, (which is incorporated by reference) discloses a mobile bearing knee prosthesis in which the tibial component includes an upstanding post and a cap provided at an upper end thereof. The cap includes a lip extending laterally outward from the post to give the cap a generally oval shape with a major axis in the A/P direction in which the post is received. The undercut cavity has a length allowing substantial movement of the post therealong in the A/P direction, and a width allowing only a minor movement of the post in the M/L direction. The insert also includes an upper cutout extending around an upper portion of the cavity which receives therein an adjacent portion of the lip.

It has been found that a wear problem for a mobile bearing knee prosthetic exists at the tibial tray post/polymeric insert interface. Generally, the mobile bearing knee prosthetic utilizes a central post on the tibial tray in concert with a polymeric tibial insert that enables a number of different possible relative motions between the tibial insert and tibial tray portion including anterior to posterior translation and rotation, rotation only, translation only, or no relative motion. During articulation, the polymeric central post contacts the cam of the femoral component. The zones of contact of the tibial tray post and the polymeric insert are both non-load bearing surfaces, however, it has been found that the articulation of the knee prosthetic causes adhesive and abrasive wear to the polymeric insert and the tibial tray post. The wear placed upon the polymeric insert from the post generates unwanted polyethylene debris.

Therefore, a need exists for a prosthetic implant that provides a strengthened, low friction, highly wear resistant surface on non-load bearing surfaces of the implant where contact occurs with another non-load bearing surface of a second prosthetic portion. Moreover, it is desirable that the post of the mobile bearing knee prosthetic employ a diffusion-hardened surface to provide reduced wear of the polymeric insert and central post and provide improved strength to the post.

### SUMMARY OF THE INVENTION

The invention provides a novel prosthetic implant that provides a strengthened, low friction, highly wear resistant surface on non-load bearing surfaces of the prosthetic device where contact occurs with another non-load bearing surface of a second prosthetic device. The contact zones of the non-load bearing surface, although not under the high stress levels and wear rate of load bearing to load bearing surfaces, benefit by the employment of a diffusion-hardened, coated surface on the non-load bearing surface of the prosthetic that contacts the second prosthetic device.

In one embodiment, the invention is directed to a mobile bearing knee system. The knee system includes a tibial tray with a proximal and distal surface, the tray is adapted to be surgically implanted on a patient's surgically cut proximal tibia. The tibial tray has a post extending up from the proximal surface of the tray. The knee system also includes a tibial insert with a proximal surface that is shaped to engage a femoral component. Additionally, the tibial insert has a distal surface that fits against and articulates with the proximal surface of the tibial tray.

The tibial insert is preferably made from a polymeric bio-compatible material, such as ultra-high molecular weight polyethylene. The proximal surface of the insert may have one or more concavities for articulating with the femoral component.

The post of the tibial tray has a diffusion-hardened surface along at least a portion of the post. Preferably, the entire post has a diffusion-hardened surface. However, the diffusion-hardened surface may cover those portions of where the post and the insert come into contact. The diffusion-hardened surface of the post in one embodiment is a thin coating of blue-black or black zirconium oxide. Also, the diffusion-hardened surface of the post also may be formed of thin coating of oxidized metal selected from one or more metals from the group consisting of hafnium, zirconium, niobium and tantalum.

The post is integrally formed as part of the tibial tray. Alternatively, all or part of the post may be separable from the tray. A locking plug member that fits a socket on the post may be used with the post. The distal surface of the tibial tray of the mobile bearing knee system may have has a stem for implantation to the patient's proximal tibia. Additionally, the distal surface of the tibial tray may have at least one spike for implantation to the patient's proximal tibia for enhanced implantation. The proximal surface of the tibial tray preferably has a diffusion-hardened surface along at least a portion of the proximal surface of the tray. In one embodiment, the diffusion-hardened surface of the tray has a thin coating of blue-black or black zirconium oxide. The coating may be formed upon all or a portion of the surface. Also, the diffusion-hardened surface of the tray may be formed of thin coating of oxidized metal selected from one or more metals from the group consisting of hafnium, zirconium, niobium and tantalum.

In another embodiment, the tibial insert has a slot on the distal surface of the insert and an opening on the proximal surface of the insert that communicates with the slot. A locking plug member can access the post from the proximal surface of the insert via the opening. The slot may have a generally cylindrically-shaped section or other shaped section that communicates with the proximal surface of the insert.

During fabrication of, the implant, the thickness of the coating of the diffusion-hardened surface of the post may vary from the thickness of the coating of the diffusion-hardened surface of the load bearing surfaces.

### BRIEF DESCRIPTION OF THE DRAWINGS

A better understanding of the invention can be obtained from the detailed description of exemplary embodiments set forth below, when considered in conjunction with the appended drawings, in which:

FIG. 1 is a perspective, exploded view of an embodiment of the apparatus of the mobile bearing knee prosthesis;

FIG. 2 is a rear, elevational and exploded view of an embodiment of the apparatus of the mobile bearing knee prosthesis illustrating the articular polymeric insert and tray portions thereof;

FIG. 3 is a sectional, elevational view of an embodiment of the apparatus of the mobile bearing knee prosthesis shown with the locking member removed;

FIG. 4 is another sectional, elevational view of an embodiment of the apparatus of the mobile bearing knee prosthesis illustrating the locking member in operating position when only rotational movement is desired;

FIG. 5 is a partial top view of an embodiment of the apparatus of the present invention showing the polymeric insert;

FIG. 6 is a partial, bottom view of an embodiment of the apparatus of the present invention showing the polymeric insert;

FIGS. 7-8 is a perspective, exploded view of another embodiment of the apparatus of the mobile bearing knee prosthesis;

FIG. 9 is a perspective, exploded view of another embodiment of the apparatus of the mobile bearing knee prosthesis; and

FIG. 10 is a perspective, exploded view of another embodiment of the apparatus of the mobile bearing knee prosthesis.

### DETAILED DESCRIPTION OF EXEMPLARY EMBODIMENTS

As used herein, “a” or “an” may mean one or more. As used herein in the claim(s), when used in conjunction with the word “comprising”, the words “a” or “an” may mean one or more than one. As used herein, “another” may mean at least a second or more.

As used herein, “diffusion-hardened surface” is defined as a type of abrasion resistant surface formed by certain specific in-situ oxidation or nitridation processes. The surface is characterized by being oxidized or nitrided relative to the substrate upon which it is situated. It is oxidized or nitrided by an in-situ oxidation or nitridation process by which oxygen or nitrogen diffuses from the surface toward the interior substrate domain. When used in reference to the underlying substrate material, it is synonymous with “surface hardened”. Also synonymously, the surface oxide or nitride layer is also referred to as “diffusion-bonded”. An oxidized or nitrided zirconium surface, as those terms are used herein, are examples of a diffusion hardened surface; other metals or metal alloys may also form diffusion-hardened surfaces by oxidation or nitridation. In all discussions herein referring to various applications and embodiments of diffusion-hardened surfaces on prosthetic devices, it should be understood that discussions with respect to oxidized surfaces apply equally to nitrided surfaces.

As used herein, “metallic” may be a pure metal or an alloy.

As used herein, “nitridation” is defined as the chemical process by which a substrate material, preferably a metal is combined with nitrogen to form the corresponding nitride.

As used herein, “zirconium alloy” is defined as any metal alloy containing zirconium in any amount greater than about 10% by weight of zirconium. Thus, an alloy in which zirconium is a minor constituent at about 10% by weight or greater is considered a “zirconium alloy” herein. Similarly, a “metal alloy” of any other named metal (e.g., a hafnium alloy or a niobium alloy; in these cases, the named metal is hafnium and niobium, respectively) is defined as any alloy containing the named metal in any amount greater than about 10% by weight.

The invention provides orthopedic implants having diffusion-hardened oxide or nitride surfaces such as oxidized zirconium or nitrided zirconium. More generally, metals or metal alloys of titanium, vanadium, niobium, hafnium and/or tantalum may be used as substrate materials to form suitable diffusion-hardened oxide surface layers. Most of the examples herein deal with zirconium or zirconium alloy substrates and surface layers of oxidized zirconium or nitrided zirconium; however, it should be understood that other metals such as hafnium, vanadium, titanium, niobium, tantalum, and their alloys, are amenable to the present invention. In order to form continuous and useful oxide or nitride coatings over the desired surface of the metal alloy prosthesis substrate, the metal alloy should preferably contain from about 80 to about 100 wt. % of the desired metal, and more preferably from about 95 to about 100 wt. %. It should be noted that in some cases, lower amount of the desired metal are possible in some cases, alloys where the desired metal is at about 10% by weight or greater may yield acceptable results. For example, an alloy of about 74 wt % titanium, about 13 wt % niobium and about 13 wt % zirconium (“Ti-13-13”) can be successfully used herein. Ti-13-13 is taught in U.S. Pat. No. 5,169,597 to Davidson et al. Thus, while levels of the desired metal of about 10% by weight or greater are known to produce acceptable results, increasing this level continuously gives progressively better results, with at least 80% by weight, and at least 95% by weight, being the preferred and most preferred levels, respectively.

In the case of either oxidized or nitrided zirconium, oxygen, niobium, and titanium, among others, may be included as common alloying elements in the alloy with often times the presence of hafnium. Yttrium may also be alloyed with the zirconium to enhance the formation of a tougher, yttria-stabilized zirconium oxide coating during the oxidation of the alloy. While oxidized or nitrided zirconium is used for illustrative purposes herein, it should be understood that the teachings apply analogously to the other possible metal candidates as well. While such zirconium containing alloys may be custom formulated by conventional methods known in the art of metallurgy, a number of suitable alloys are commercially available. In the case of oxidized zirconium. some commercial alloys include, among others Zircadyne 705, Zircadyne 702, and Zircalloy.

The base metal and metal alloys are cast or machined by conventional methods to the shape and size desired to obtain a suitable prosthesis substrate. The substrate is then subjected to process conditions which cause the in situ formation of a tightly adhered, diffusion-bonded coating of zirconium oxide or zirconium nitride on its surface. The term “diffusion-hardened” and “diffusion-bonded” are used in reference to the desired oxides or nitrides because the formation of these particular surfaces is characterized by the diffusion of oxygen or nitrogen from the surface towards the interior (i.e., approaching the unoxidized substrate, native metal or metal alloy). It is believed that this diffusion of oxygen or nitrogen is what imparts the high strength and high wear resistance to these surfaces. The process conditions for formation include, for instance, air, steam, or water oxidation or oxidation in a salt bath. These processes ideally provide a thin, hard, dense, low friction, wear-resistant zirconium nitride or blue-black or black wear-resistant zirconium oxide film or coating of thicknesses typically on the order of several microns (10-6 meter) on the surface of the prosthesis substrate. Below this coating, diffused oxygen or nitrogen from the oxidation or nitridation process increases the hardness and strength of the underlying substrate metal.

The air, steam and water oxidation processes are described for zirconium and zirconium alloys in now-expired U.S. Pat. No. 2,987,352 to Watson, the teachings of which are incorporated by reference as though fully set forth. These methods may also be applied to metals and alloys of hafnium, titanium, vanadium, niobium, and tantalum. In the case of zirconium or zirconium alloy, the air oxidation process provides a firmly adherent black or blue-black layer of zirconium oxide of highly oriented monoclinic crystalline form. If the oxidation process is continued to excess, the coating will whiten and separate from the metal substrate. The oxidation step may be conducted in either air, steam or hot water. For convenience, the metal prosthesis substrate may be placed in a furnace having an oxygen-containing atmosphere (such as air) and typically heated at 700° F.-1100° F. up to about 6 hours. However, other combinations of temperature and time are possible. When higher temperatures are employed, the oxidation time should be reduced to avoid the formation of the white oxide.

The oxide layer should range in thickness from about 1 to about 20 microns; however, a range of from about 1 to about 5 microns is preferred. The overall average thickness can be controlled by the parameters of time and temperature. For example, furnace air oxidation at 1000° F. for 3 hours will form an oxide coating on Zircadyne 705 about 2-3 microns thick, oxidation at 1175° F. for 1 hour results in an overall average oxide coating of about 4-5 microns thick, and oxidation at 1175° F. for 3 hours results in an overall average oxide coating of about 10-11 microns thick. As additional examples, one hour at 1300° F. will form an oxide coating about 14 microns in thickness, while 21 hours at 1000° F. will form an oxide coating thickness of about 9 microns. Using different combinations of oxidation times and higher oxidation temperatures will increase or decrease this thickness, but higher temperatures and longer oxidation times may compromise coating integrity, depending upon the nature of the substrate and other factors. For thicker coatings of oxide, some trial and error may be necessary. Of course, because in the usual case only a thin oxide is necessary on the surface, only very small dimensional changes, typically less than 10 microns over the thickness of the prosthesis, will result. In general, thinner coatings (1-4 microns) have better attachment strength.

One of the salt-bath methods that may be used to apply the oxide coatings to the metal alloy prosthesis, is the method of U.S. Pat. No. 4,671,824 to Haygarth (the '824 patent), the teachings of which are incorporated by reference as though fully set forth. In the case of oxidized zirconium, the salt-bath method provides a similar, slightly more abrasion resistant blue-black or black zirconium oxide coating. The method requires the presence of an oxidation compound capable of oxidizing zirconium in a molten salt bath. The molten salts include chlorides, nitrates, cyanides, and the like. The oxidation compound, sodium carbonate, is present in small quantities, up to about 5 wt %. The addition of sodium carbonate lowers the melting point of the salt. As in air oxidation, the rate of oxidation is proportional to the temperature of the molten salt bath and the '824 patent prefers the range 550° C. -800° C. (1022° F. -1470° F.). However, the lower oxygen levels in the bath produce thinner coatings than for furnace air oxidation at the same time and temperature. A salt bath treatment at 1290° F. for four hours produces an oxide coating thickness of roughly 7 microns.

Whether air oxidation in a furnace or salt bath oxidation is used, the oxide coatings are quite similar in hardness. For example, if the surface of a wrought Zircadyne 705 (Zr, 2-3 wt. % Nb) prosthesis substrate is oxidized, the hardness of the surface shows a dramatic increase over the 200 Knoop hardness of the original metal surface. The surface hardness of the resulting blue-black zirconium oxide surface following oxidation of Zircadyne 705 by either the salt bath or air oxidation process is approximately 1700-2000 Knoop hardness.

In the case of nitridation of zirconium and zirconium alloys, an analogous procedure is used. As in the oxide case, the nitride layer should range in thickness from about 1 to about 20 microns; however, a range of from about 1 to about 5 microns is preferred. Even though air contains about four times as much nitrogen as oxygen, when zirconium or a zirconium alloy is heated in air as described above, the oxide coating is formed in preference to the nitride coating. This is because the thermodynamic equilibrium favors oxidation over nitridation under these conditions. Thus, to form a nitride coating the equilibrium must be forced into favoring the nitride reaction. This is readily achieved by elimination of oxygen and using a nitrogen or ammonia atmosphere instead of air or oxygen when a gaseous environment (analogous to “air oxidation”) is used. In order to form a zirconium nitride coating of about 5 microns in thickness, the zirconium or zirconium alloy prosthesis should be heated to about 800° C. for about one hour in a nitrogen atmosphere. Thus, apart from the removal of oxygen (or the reduction in oxygen partial pressure), or increasing the temperature, conditions for forming the zirconium nitride coating do not differ significantly from those needed to form the blue-black or black zirconium oxide coating. Any needed adjustment would be readily apparent to one of ordinary skill in the art.

When a salt bath method is used to produce a nitride coating, then the oxygen-donor salts should be replaced with nitrogen-donor salts, such as, for instance cyanide salts. Upon such substitution, a nitride coating may be obtained under similar conditions to those needed for obtaining an oxide coating. Such modifications as are necessary, may be readily determined by those of ordinary skill in the art. Alternatively, the zirconium nitride may be deposited onto the zirconium or zirconium alloy surface via standard physical or chemical vapor deposition methods, including those using an ion-assisted deposition method. It is preferred that the physical or chemical vapor deposition methods be carried out in an oxygen-free environment. Techniques for producing such an environment are known in the art, for instance the bulk of the oxygen may be removed by evacuation of the chamber and the residual oxygen may be removed with an oxygen scavenger.

When the zirconium or zirconium alloy is provided with a zirconium porous bead, zirconium wire mesh surface, or textured surface, then this surface layer can also be coated with zirconium oxide or nitride, as the case may be, to provide protection against metal ionization in the body.

These diffusion-bonded, low friction, highly wear resistant oxidized or nitrided zirconium coatings are grown in-situ and used on the surfaces of orthopedic implants subject to conditions of wear.

Mobile Bearing Knee Prosthetic—Tibial Tray with Post

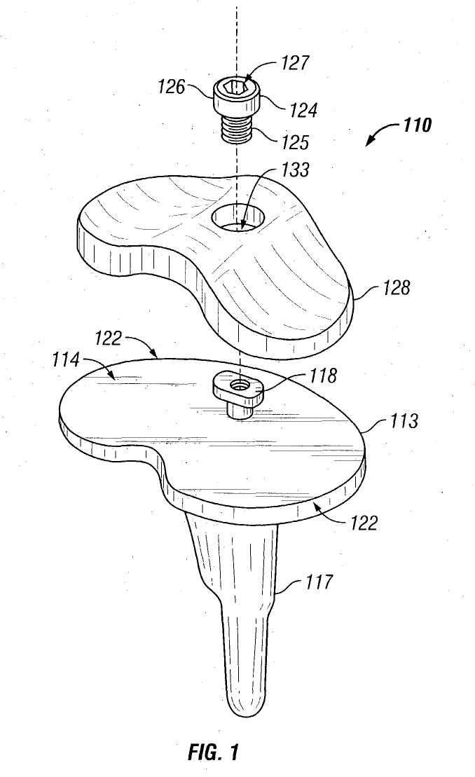

Now referring to FIGS. 1-6, the illustrations show generally an embodiment of the apparatus of the mobile bearing knee prosthesis designated generally by the numeral 110 in FIGS. 1, 3 and 4.

Mobile bearing knee prosthesis 110 is placed upon a patient's surgically cut proximal tibia 111 at a surgically cut proximal surface 112 that is preferably flat. This enables a tray 113 to be mounted to the proximal tibia 111 at the surface 112 as shown in FIGS. 3 and 4. Tray 113 has a flat proximal surface 114 and a generally flat distal surface 115 that mates with and faces the surgically prepared surface 112 as shown in FIGS. 3 and 4. The tray 113 can provide a plurality of spikes 116 and a stem 117 for enhancing implantation to the patient's proximal tibia 111.

The proximal surface 114 of tray 113 provides a post 118 having an internally threaded socket 119. The post 118 employs a diffusion-hardened surface. Preferably the entire surface of the post employs the diffusion-hardened surface. However, the post may only or partially employ the diffusion-hardened surface about the surface of the post in the contact zones with the tibial insert 128 which improves wear resistance. The contact zones are those surfaces of the post that contact or interface with the tibial insert 128. In one embodiment, a zirconium oxide coating is formed on the post 118 though oxidation of a zirconium or zirconium-based alloy. The formation of the zirconium oxide coating may be formed as discussed herein. After the oxide coating on the post 118 is formed, the oxide coating may be polished to exhibit a mirror-like finish. The post's diffusion-hardened surface results in added strength to the post. Additionally, reduction of wear to the post will be achieved over other metals, such as cobalt chrome, that are utilized for the manufacture of a knee prosthetic. Additionally, the locking member 124 may employ a diffusion-hardened surface about its periphery. Such a diffusion-hardened surface is especially useful where the surface of the locking member 124 contacts the insert 128.

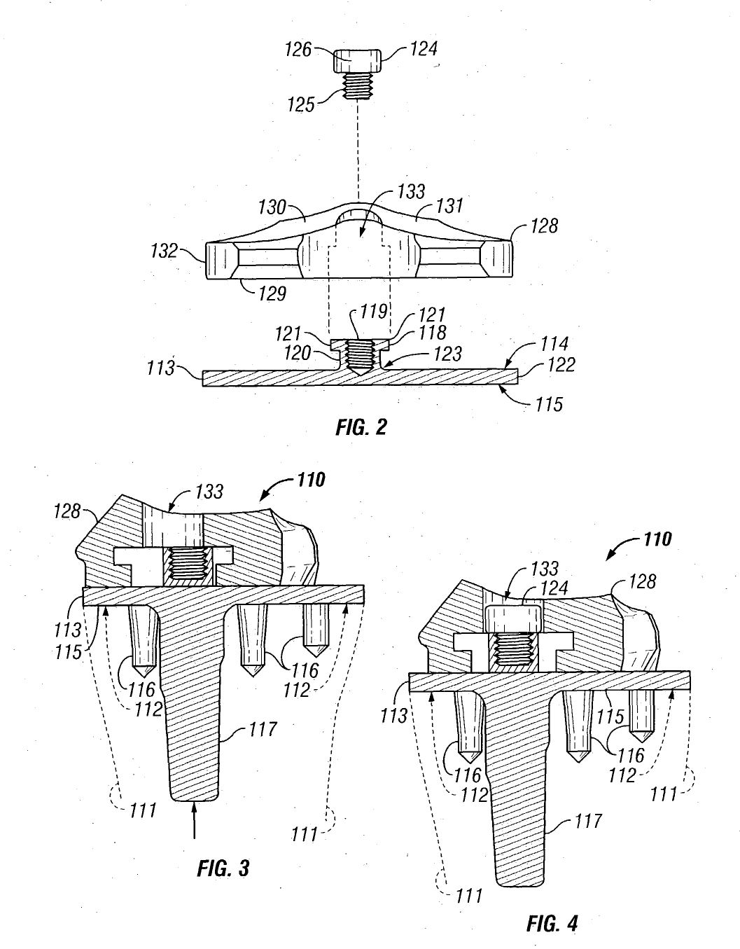

Post 118 is comprised of a generally cylindrically-shaped smaller diameter section 120 and an enlarged flange 121 that mounts to the top of cylindrically-shaped 120 as shown in FIGS. 2. Tray 113 has a periphery 122. A recess 123 is provided in between the proximal surface 114 of tray 113 and flange 121.

A locking member 124 forms a removable connection with the socket 119. Locking member 124 has an externally cylindrical section 125 that provides threads that correspond to the threads of internally socket 119 so that the locking member 124 can be threaded into the socket 119 as shown in FIG. 4. Locking member 124 includes an enlarged cylindrically-shaped head 126 having a tool receptive socket 127 such as a hexagonal socket for example.

A polymeric insert 128 provides a vertical channel 133 that can be placed in communication with post 118 as shown in FIGS. 3 and 4. Insert 128 provides a preferably flat distal surface 129 that communicates with the flat proximal surface 114 of tray 113. A pair of spaced apart concavities 130, 131 on the distal surface of the insert 128 are provided for defining articulation surfaces that cooperate with correspondingly shaped articulating surface on a patient's femur or femoral component. The insert 128 has a periphery 132 that generally corresponds in shape to the periphery 122 of tray 113. Also, the proximal surface of the tibial tray employs a diffusion-hardened surface. For example, the formation of a coating of oxidized zirconium on the proximal surface of the tibial tray provides reduced wear of the tray and the polymeric mating surfaces.

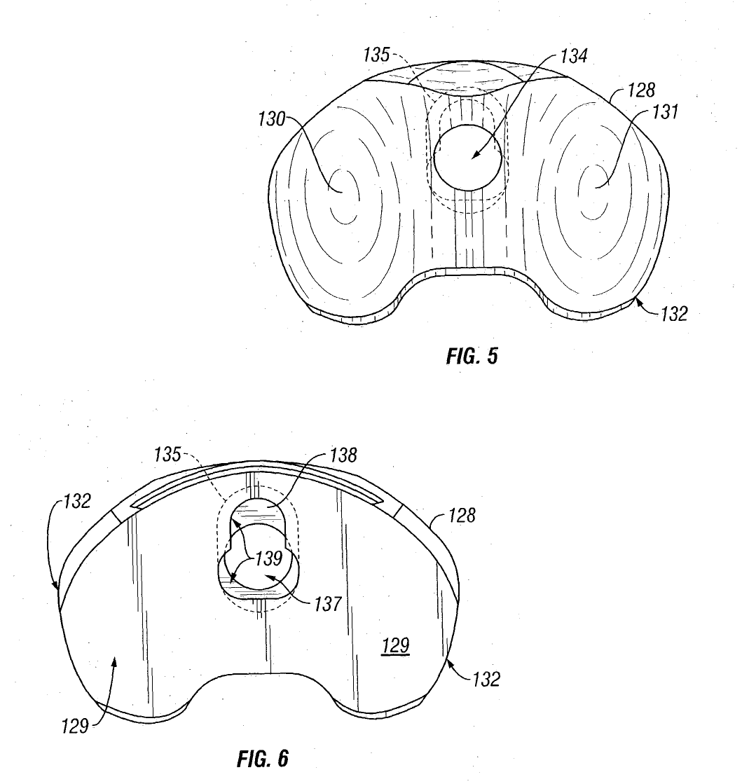

Vertical channel 133 is comprised of a number of sections that are specially shaped to interact with the post 118 and locking member 124. Vertical channel 133 thus includes a proximal, cylindrically-shaped section 134, an oval shaped slot 135, and a distal opening 136. The distal opening 136 includes a generally oval section 137 and a somewhat half oval section 138. Flat surfaces 139, 140 are positioned at the top of and at the bottom of the oval shaped slot 135 as best seen in FIGS. 5-6. The cylindrically-shaped head 126 of the locking member 124 closely fits the cylindrically-shaped section 136.

In order to assemble insert 128 to tray 113, the distal surface of 129 of insert 128 is placed next to and generally parallel to the proximal surface 114 of tray 113. Post 118 is aligned with vertical channel 133 of insert 128. During assembly of insert 128 to tray 113, the post 118 is shaped to enter the oval opening portion 137 of distal opening 136. Once the distal surface 129 of insert 128 meets proximal surface 114 of tray 113, flange 121 aligns with oval shaped slot 135 of vertical channel 133. After such assembly, insert 128 is held in position by post 118.

The polymeric insert 128 may optionally be provided with a post extending from the distal surface of the insert 128. The post extending from the polymeric insert may be used to provide posterior stabilization with a femoral component. This post articulates with a femoral component having a cam and box.

In addition to the diffusion hardened surface about the slot, optionally the proximal surface 114 may be made with a diffusion hardened surface or optionally the entire tibial tray 113 may be made with a diffusion hardened surface.

Mobile Bearing Knee Prosthetic—Tibial Tray with Slot

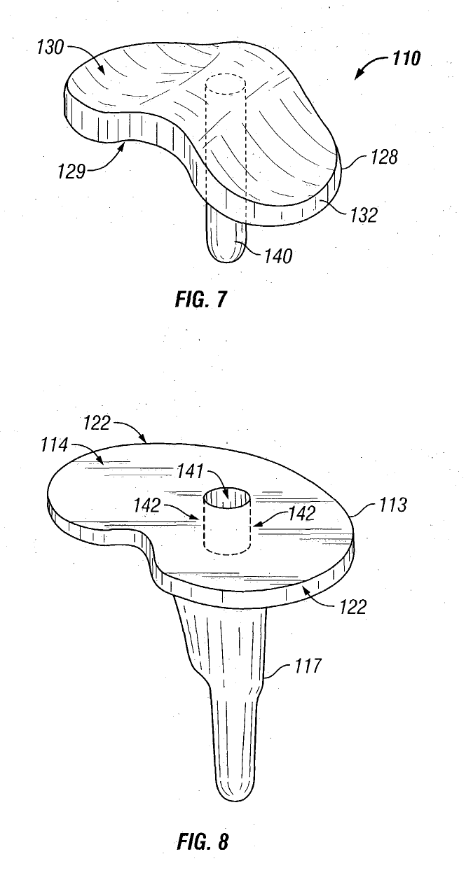

Now referring to FIGS. 7 and 8, an alternate embodiment of the mobile bearing knee prosthesis is shown. Insert 128 provides a preferably flat distal surface 129 that communicates with the flat proximal surface 114 of tray 113. A pair of spaced apart concavities 130, 131 on the distal surface of the insert 128 are provided for defining articulation surfaces that cooperate with correspondingly shaped articulating surface on a patient's femur or femoral component. The insert 128 has a periphery 132 that generally corresponds in shape to the periphery 122 of tray 113.

A polymeric insert 128 has a post 140 extending from the proximal surface of the insert 128. The post 140 is preferably integrally formed with the polymeric insert 128. However, the post 140 may be detachable. Preferably the post 140 is made from the same material of the tray 140, but may be made from other material, such as metals other polymers. The tibial tray 113 has a slot 141 in which the post 140 of the polymeric insert articulates. Preferably, the shape of the slot 141 allows the post 140 to rotate or articulate within the slot 141. For illustration, but not limitation, various shapes may be utilized for the post 140 including partially spherical, elliptical, conical, cylindrical and combinations thereof. The slot 141 of the tibial tray 113 employs a diffusion-hardened surface along the surface of the slot wall 142. The slot wall 142 may be flat, concave, convex or any other useful shape. Preferably the entire surface of the slot wall 142 employs the diffusion-hardened surface, especially along the surface of the slot wall 142 where the post 140 of the insert articulates with the slot 141. In one embodiment, a zirconium oxide coating is formed on the surface of the slot wall 142 though oxidation of a zirconium or zirconium-based alloy. After the oxide coating on the slot wall 142 is formed, the oxide coating may be polished to exhibit a mirror-like finish.

The polymeric insert 128 may optionally be provided with a post extending from the distal surface of the insert. The post extending from the polymeric insert may be used to provide posterior stabilization with a femoral component. This post articulates with a femoral component having a cam and box.

In addition to the diffusion hardened surface about the post, optionally the proximal surface 114 may be made with a diffusion hardened surface or optionally the tibial tray 113 may be made with a diffusion hardened surface.

Mobile Bearing Knee Prosthetic—Tray with Channels and Rails

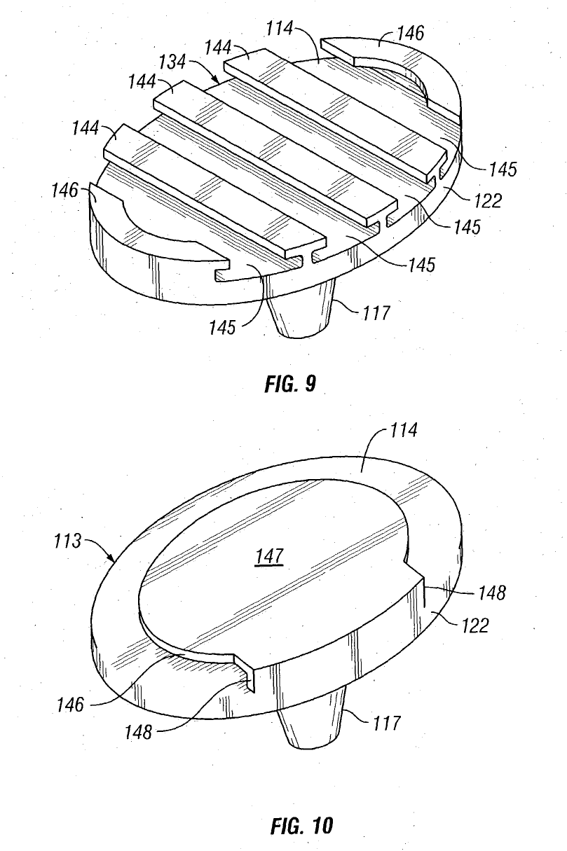

Now referring to FIG. 9, an alternative embodiment of the mobile bearing knee prosthesis is shown. FIG. 9, however, shows only the tibial tray 113. Tray 113 has a periphery 122. The tray 113 can provide a plurality of spikes (not shown) and a stem 117 for enhancing implantation to the patient's proximal tibia. A tibial tray 113 has rails 144 extending from the proximal surface 114 thereby forming channels 145. A polymeric insert has a corresponding structure such that the channels and rails of the tibial tray 113 interlock with the channels and rails of the polymeric insert. The outer most channels 146 are contoured similar to the sides of the tray 113.

The channels and rails of the polymeric insert and the tibial tray 113 interact with each other such that the insert and tray 113 articulate in a linear forward and backward motion.

The tibial tray 113 employs a diffusion-hardened surface along the surface of the rails 144 and along the surface of the channels 144. The channel wall 144 may be flat, concave, convex or any other useful shape. In one embodiment, a zirconium oxide coating is formed on the surface of the rails 144 and the channels 145 though oxidation of a zirconium or zirconium-based alloy. After the oxide coating on is formed, the oxide coating may be polished to exhibit a mirror-like finish.

The polymeric insert may optionally be provided with a post extending from the distal surface of the insert. The post extending from the polymeric insert may be used to provide posterior stabilization with a femoral component. This post articulates with a femoral component having a cam and box.

In addition to the diffusion hardened surface about the rails and channels, optionally the entire tibial tray 113 may be made with a diffusion hardened surface.

Mobile Bearing Knee Prosthetic—Tray with Circular Shaped Surface

Now referring to FIG. 10, an alternative embodiment of the mobile bearing knee prosthesis shown. FIG. 10, however, shows only the tibial tray 113. Tray 113 has a periphery 122. The tray 113 can provide a plurality of spikes (not shown) and a stem 117 for enhancing implantation to the patient's proximal tibia. In one embodiment, the tray 113 has a raised cylindrical shaped surface and the proximal surface of the insert has a depressed surface generally corresponding to the shape of the raised cylindrical shaped surface. In an alternative embodiment (not shown), the tray has a depressed cylindrical shaped surface and the tray has a raised cylindrical shaped surface corresponding to the depressed cylindrical shaped surface. Such a structure allows the tray 113 and the insert to rotate with one another. The tray 113 has two stops 148 where the insert will contact that tibial tray to prevent further rotation of the insert.

In either embodiment, the cylindrical shaped surface of the tray 113 employs a diffusion-hardened surface. In one embodiment, a zirconium oxide coating is formed on the shaped surface though oxidation of a zirconium or zirconium-based alloy. After the oxide coating on the circular shaped surface is formed, the oxide coating may be polished to exhibit a mirror-like finish.

The polymeric insert may optionally be provided with a post extending from the distal surface of the insert. The post extending from the polymeric insert may be used to provide posterior stabilization with a femoral component. This post articulates with a femoral component having a cam and box.

In addition to the diffusion hardened surface about the surface of the circular shaped surface, optionally the entire tibial tray 113 may be made with a diffusion hardened surface.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the invention described in the specification. As one of ordinary skill in the art will readily appreciate from the disclosure of the present invention, devices, means, metals and alloys existing or later to be developed that perform substantially the same function or achieve substantially the same result as the corresponding embodiments described herein may be utilized according to the present invention. Accordingly, the appended claims are intended to include within their scope such devices, means, and metals and alloys.

What is claimed is:

1. A mobile bearing knee system comprising:
a tibial tray having a distal surface adapted to be surgically implanted on a patient's surgically cut proximal tibia, the tibial tray having a proximal surface with a post extending up from the proximal surface of the tray; and
a tibial insert having a proximal surface that is shaped to engage a femoral component, the insert having a distal surface that fits against and articulates with the proximal surface of the tibial tray;
wherein the post having a diffusion-hardened surface along at least a portion of the post where the post communicates with the tibial insert.

2. The mobile bearing knee system of claim 1, wherein the diffusion-hardened surface of the post is a thin coating of blue-black or black zirconium oxide.

3. The mobile bearing knee system of claim 1, wherein the diffusion-hardened surface of the post is a thin coating of oxidized metal selected from one or more metals from the group consisting of hafnium, zirconium, niobium and tantalum.

4. The mobile bearing knee system of claim 1, wherein the polymeric biocompatible material is ultra-high molecular weight polyethylene.

5. The mobile bearing knee system of claim 1, wherein the distal surface of the tibial tray having a stem for implantation to the patient's proximal tibia.

6. The mobile bearing knee system of claim 1, wherein the distal surface of the tibial tray having at least one spike for implantation to the patient's proximal tibia.

7. The mobile bearing knee system of claim 1, wherein the proximal surface of the insert having one or more concavities for articulating with the femoral component.

8. The mobile bearing knee system of claim 1, wherein all or part of the post is separable from the tray.

9. The mobile bearing knee system of claim 1, further comprising a locking plug member that fits a socket on the post.

10. The mobile bearing knee system of claim 9, wherein the insert having a slot on the distal surface of the insert, an opening on the proximal surface of the insert that communicates with the slot, and wherein the locking plug member can access the post from the proximal surface of the insert via the opening.

11. The mobile bearing knee system of claim 10, wherein the slot has a generally cylindrically-shaped section that communicates with the proximal surface of the insert.

12. The mobile bearing knee system of any one of claims 1-11, wherein the proximal surface of the tibial tray having a first diffusion-hardened surface along at least a portion of the proximal surface of the tray.

13. The mobile bearing knee system of claim 12, wherein the diffusion-hardened surface of the proximal surface of the tibial tray is a thin coating of blue-black or black zirconium oxide.

14. The mobile bearing knee system of claim 12, wherein the diffusion-hardened surface of the proximal surface of the tibial tray is a thin coating of oxidized metal selected from one or more metals from the group consisting of hafnium, zirconium, niobium and tantalum.

15. The mobile bearing knee system of claim 1, wherein the post is partially spherical, elliptical, conical, or cylindrical in shape.

16. The mobile bearing knee system of claim 1, wherein the proximal surface having a diffusion-hardened surface along at least a portion of the proximal surface.

17. A mobile bearing knee system comprising:
a tibial tray having a distal surface adapted to be surgically implanted on a patient's surgically cut proximal tibia, the tibial tray having a proximal surface having a slot; and
a tibial insert having a proximal surface that is shaped to engage a femoral component and a distal surface that fits against and articulates with the proximal surface of the tibial tray, the tibial insert having a proximal surface with a post that communicates with the slot;
wherein the slot having a diffusion-hardened surface along at least a portion of the slot where the post communicates with the slot.

18. The mobile bearing knee system of claim 17, wherein the diffusion-hardened surface of the slot is a thin coating of blue-black or black zirconium oxide.

19. The mobile bearing knee system of claim 17, wherein the diffusion-hardened surface of the slot is a thin coating of oxidized metal selected from one or more metals from the group consisting of hafnium, zirconium, niobium and tantalum.

20. The mobile bearing knee system of claim 17, wherein the polymeric biocompatible material is ultra-high molecular weight polyethylene.

21. The mobile bearing knee system of claim 17, wherein the distal surface of the tibial tray having a stem for implantation to the patient's proximal tibia.

22. The mobile bearing knee system of claim 17, wherein the distal surface of the tibial tray having at least one spike for implantation to the patient's proximal tibia.

23. The mobile bearing knee system of claim 17, wherein the proximal surface of the insert having one or more concavities for articulating with the femoral component.

24. The mobile bearing knee system of claim 17, wherein the post is partially spherical, elliptical, conical, or cylindrical in shape.

25. A mobile bearing knee system comprising:
a tibial tray having a distal surface adapted to be surgically implanted on a patient's surgically cut proximal tibia and a proximal surface having at least one rail on the proximal surface; and
a tibial insert having a proximal surface that is shaped to engage a femoral component and a distal surface that fits against and articulates with the proximal surface of the tibial tray, the tibial insert having a proximal surface with at least one channel that communicates with the at least one rail;
wherein the rail having a diffusion-hardened surface along at least a portion of the rail where the rail communicates with the channel.

26. The mobile bearing knee system of claim 25, wherein the diffusion-hardened surface of the rail is a thin coating of blue-black or black zirconium oxide.

27. The mobile bearing knee system of claim 25, wherein the diffusion-hardened surface of the rail is a thin coating of oxidized metal selected from one or more metals from the group consisting of hafnium, zirconium, niobium and tantalum.

28. The mobile bearing knee system of claim 25, wherein the polymeric biocompatible material is ultra-high molecular weight polyethylene.

29. The mobile bearing knee system of claim 25, wherein the distal surface of the tibial tray having a stem for implantation to the patient's proximal tibia.

30. The mobile bearing knee system of claim 25, wherein the distal surface of the tibial tray having at least one spike for implantation to the patient's proximal tibia.

31. The mobile bearing knee system of claim 25, wherein the proximal surface of the insert having one or more concavities for articulating with the femoral component.

32. The mobile bearing knee system of claim 25, wherein all of the surface of the at least one rail has a diffusion-hardened surface.

33. A mobile bearing knee system comprising:
a tibial tray having a distal surface adapted to be surgically implanted on a patient's surgically cut proximal tibia and a proximal surface having at least one channel; and
a tibial insert having a proximal surface that is shaped to engage a femoral component and a distal surface that fits against and articulates with the proximal surface of the tibial tray, the tibial insert having a proximal surface with at least one rail that communicates with the at least one channel;
wherein the at least one channel having a diffusion-hardened surface along at least a portion of the channel where the rail communicates with the channel.

34. The mobile bearing knee system of claim 33, wherein the diffusion-hardened surface of the channel is a thin coating of blue-black or black zirconium oxide.

35. The mobile bearing knee system of claim 33, wherein the diffusion-hardened surface of the channel is a thin coating of oxidized metal selected from one or more metals from the group consisting of hafnium, zirconium, niobium and tantalum.

36. The mobile bearing knee system of claim 33, wherein the polymeric bio-compatible material is ultra-high molecular weight polyethylene.

37. The mobile bearing knee system of claim 33, wherein the distal surface of the tibial tray having a stem for implantation to the patient's proximal tibia.

38. The mobile bearing knee system of claim 33, wherein the distal surface of the tibial tray having at least one spike for implantation to the patient's proximal tibia.

39. The mobile bearing knee system of claim 33, wherein the proximal surface of the insert having one or more concavities for articulating with the femoral component.

40. The mobile bearing knee system of claim 33, wherein all of the surface of the at least one channel has a diffusion-hardened surface.

41. A mobile bearing knee system comprising:
a tibial tray having a distal surface adapted to be surgically implanted on a patient's surgically cut proximal tibia and a proximal surface having a shaped circular surface;
a tibial insert having a proximal surface that is shaped to engage a femoral component and a distal surface that fits against and articulates with the proximal surface of the tibial tray, the tibial insert having a shaped circular surface that communicates with the tibial tray's shaped circular surface;
wherein the tibial tray's shaped circular surface having a diffusion-hardened surface.

42. The mobile bearing knee system of claim 41, wherein the diffusion-hardened surface is a thin coating of blue-black or black zirconium oxide.

43. The mobile bearing knee system of claim 41, wherein the diffusion-hardened surface is a thin coating of oxidized metal selected from one or more metals from the group consisting of hafnium, zirconium, niobium and tantalum.

44. The mobile bearing knee system of claim 41, wherein the polymeric bio-compatible material is ultra-high molecular weight polyethylene.

45. The mobile bearing knee system of claim 41, wherein the distal surface of the tibial tray having a stem for implantation to the patient's proximal tibia.

46. The mobile bearing knee system of claim 41, wherein the distal surface of the tibial tray having at least one spike for implantation to the patient's proximal tibia.

47. The mobile bearing knee system of claim 41, wherein the proximal surface of the insert having one or more concavities for articulating with the femoral component.
